(19) United States
(12) Patent Application Publication
(10) Pub. No.: US 2019/0307920 A1
(43) Pub. Date: Oct. 10, 2019

(54) Self-Securing Biological Tissue Graft

(71) Applicant: Scendia Biologics, LLC, Orlando, FL (US)

(72) Inventors: Ryan Phillips, Orlando, FL (US); David Matlock, Orlando, FL (US); Sijo Parekattil, Clermont, FL (US); Jamin Brahmbhatt, Clermont, FL (US)

(21) Appl. No.: 16/379,739

(22) Filed: Apr. 9, 2019

Related U.S. Application Data
(60) Provisional application No. 62/655,205, filed on Apr. 9, 2018.

(51) Int. Cl. A61L 27/36; A61K 35/50

(52) U.S. Cl. CPC A61L 27/3604; A61F 2/0077; A61L 27/3662; A61K 35/50

(57) ABSTRACT

An example biological tissue graft includes a body formed from a flexible biological tissue, the body having a first end and a second end opposite the first end. The biological tissue graft also includes an aperture positioned in the second end of the body that is sized to receive the first end of the body therethrough.

### CROSS REFERENCE TO RELATED APPLICATIONS

This application claims priority to U.S. Provisional Patent Application No. 62/655,205, filed on Apr. 9, 2018 and entitled “Self-Securing Biological Tissue Graft,” which is incorporated herein by reference its entirety.

### BACKGROUND

Amniotic tissue grafts are created by processing the amniotic membrane from a donated placenta following caesarean section births. The amniotic membrane is the inner layer of the placenta that surrounds a baby during pregnancy and is comprised of the epithelial, amnion and chorion layers. The amniotic membrane is the only natural neutral tissue found in nature, which protects the baby during pregnancy and prevents rejection from the mother's immune system. This inherent characteristic of the amniotic membrane has led it to be used in medicine since the early 1900s. Traditionally, amniotic membranes have been used as grafts to cover wounds and assist with the healing of burns. In modern times, it has become common to use tissue grafts formed from the amniotic membrane as an anatomical barrier in surgical procedures to prevent scar adhesion between two tissues; particularly as an on-lay or wrap to cover and protect certain anatomical bodies such as a tendon, ligament, muscle, nerve, dura, vein, artery, spermatic cord, or other tissues.

Various surgical procedures include securing the tissue graft in place to prevent migration of the tissue graft. When a tissue graft is wrapped around an approximately cylindrical object, a suture is frequently used to prevent unraveling. This may add time to the procedure, which can be undesirable in some situations. Further, amniotic tissue grafts can be relatively fragile, and may tear when placing a suture through the graft. When a tear occurs, the graft cannot be securely held in place and migration risk increases. In other existing implementations, a fibrin or biocompatible glue is sometimes used to tack a graft to itself or surrounding tissues. However, this application can also be difficult to implement for some procedures. For example, precise application of the glue while holding the tissue graft in place may be difficult. The glue may also cause unwanted outcomes in some cases. For instance, leaking of the glue may cause unwanted adherence of the graft to surrounding tissues.

Moreover, the ability to glue or suture a tissue graft may not always be feasible based on the surgical procedure. Complex anatomy can prevent the ability to tack the graft properly. Further, small wound exposure can limit the ability to access the graft and tack it properly. Thus, glue and sutures may have a limited scope of use, and there is a need for improved structures and methods for securing a tissue graft in a desired location.

### BRIEF DESCRIPTION OF THE DRAWINGS

Features, aspects, and advantages of the presently disclosed technology may be better understood with regard to the following description, appended claims, and accompanying drawings, as listed below. A person skilled in the relevant art will understand that the features shown in the drawings are for purposes of illustrations, and variations, including different and/or additional features and arrangements thereof, are possible.

FIG. 1A is a perspective view of a self-securing biological tissue graft, according to an example implementation.

FIG. 1B is a perspective view of the self-securing biological tissue graft of FIG. 1A wrapped around an anatomical body, according to an example implementation.

FIG. 1C is an end view of the self-securing biological tissue graft wrapped around the anatomical body of FIG. 1B, according to an example implementation.

FIG. 2 is a perspective view of a self-securing biological tissue graft, according to another example implementation.

FIG. 3 is a perspective view of a self-securing biological tissue graft, according to another example implementation.

FIG. 4 is a perspective view of a self-securing biological tissue graft, according to another example implementation.

FIG. 5 is a flowchart illustrating a method for implanting a biological tissue graft, according to an example implementation.

The drawings are for the purpose of illustrating example embodiments, but those of ordinary skill in the art will understand that the technology disclosed herein is not limited to the arrangements and/or instrumentality shown in the drawings.

### DETAILED DESCRIPTION

### I. Overview

Embodiments discussed herein relate to a self-securing, biological tissue graft, such as a self-securing biological tissue graft formed from amniotic tissue. Use of such a self-securing biological tissue graft may facilitate the implantation of the graft in such a way that reduces the likelihood of displacement or migration of the tissue graft from its desired position.

In some embodiments, for example, a biological tissue graft is provided including a body formed from a flexible biological tissue, the body having a first end and a second end opposite the first end. The biological tissue graft also includes an aperture positioned in the second end of the body and sized to receive the first end of the body therethrough.

In another aspect, a method for implanting a biological tissue graft during a surgical procedure is provided, where the biological tissue graft comprises a body formed from a flexible biological tissue, the body having a first end, a second end opposite the first end, and an aperture positioned in the second end of the body and sized to receive the first end of the body therethrough. The method includes positioning the biological tissue graft adjacent to an anatomical body, surrounding the anatomical body with the biological tissue graft, and inserting the first end of the biological tissue graft through the aperture.

While some examples described herein may refer to functions performed by given actors such as a surgeon and/or other entities, it should be understood that this is for purposes of explanation only. The claims should not be interpreted to require action by any such example actor unless explicitly required by the language of the claims themselves.

In the Figures, identical reference numbers identify generally similar, and/or identical, elements. Many of the details, dimensions, angles and other features shown in the Figures are merely illustrative of particular embodiments of the disclosed technology. Accordingly, other embodiments can have other details, dimensions, angles and features without departing from the spirit or scope of the disclosure. In addition, those of ordinary skill in the art will appreciate that further embodiments of the various disclosed technologies can be practiced without several of the details described below.

### II. Self-Securing Biological Tissue Graft Implementations

Embodiments discussed herein provide for a self-securing biological tissue graft. For example, an amniotic tissue graft may be formed with an integrated belt and buckle arrangement that allows the tissue graft to be secured to itself, which may prevent migration or unraveling of the tissue graft once it is positioned adjacent to an anatomical body. In some embodiments, this arrangement may eliminate the need to fixate the graft with glue or sutures, or alternatively, may improve such existing implementations.

Accordingly, advantages of the self-securing biological tissue graft discussed herein may include, for example, reducing the time needed to place and secure a tissue graft, such as an amniotic tissue graft, in the surgical theater, reducing operative costs by eliminating unneeded glues and sutures, preventing possible patient allergic or autoimmune reactions to glues or sutures, and preventing tearing of the amniotic graft while placing a suture through the graft to secure it around a body. Further advantages will become apparent to those of skill in the art from a study of the following description and the accompanying drawings.

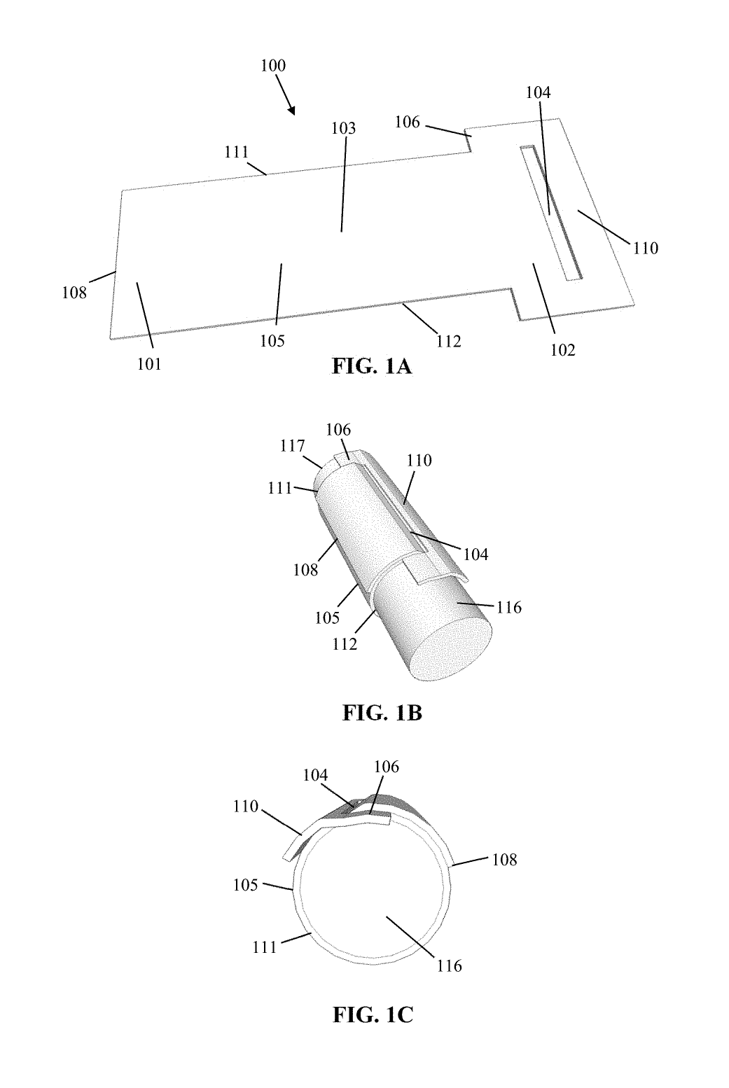

One embodiment of a self-securing biological tissue graft 100 is illustrated in FIG. 1-A (perspective top view), FIG. 1-B (wrapped view), and FIG. 1-C (end view). The self-securing biological tissue graft 100 of FIGS. 1A-1C may be, for example, an amniotic tissue wrap 100 for placement around an anatomical body 116, as will be discussed below. The wrap 100 consists of a body 103 having a first end 101 and a second end 102 opposite the first end 101. The wrap 100 may be formed from a flexible sheet of biological material that may be rolled or twisted without fracturing, for example. An aperture 104 may be positioned in the second end 102 of the body 103, and the aperture 104 may be sized to receive the first end 101 of the body 103 therethrough.

For example, the first end 101 of the amniotic wrap 100 may include a leading edge 108, as shown in FIG. 1A. Further, in some embodiments as otherwise described herein, the aperture 104 may be formed as a slit 104, although other shapes and arrangements for the aperture 104 are also possible. As shown in FIG. 1A, the slit 104 may have an opening width that is wider than the first width of the leading edge 108. Moreover, the body 103 may include a belt portion 105 positioned between the first end 101 and the second end 102, and the opening width of the slit 104 may also be wider than a belt width of the belt portion 105.

In some implementations, the second end 102 may include a buckle portion 110 that includes the slit 104. The buckle portion 110 may include a buckle wing 106 that extends from both a first side edge 111 and a second side edge 112 of the body 103. The wings 106 may each extend wider than the belt portion 105, and may frame the slit 104 therebetween.

FIGS. 1B and 1C illustrate an example of the amniotic wrap 100 in use around an anatomical body 116 that is to be protected by the wrap 100. For example, the anatomical body 116 may be a tendon, ligament, muscle, nerve, dura, vein, artery, spermatic cord, among other possibilities. In use, the amniotic wrap 100 may be positioned adjacent to the anatomical body 116. As shown in FIGS. 1B-1C, the wrap 100 is passed underneath and around the anatomical body 116 to circumferentially surround the anatomical body 116 with the wrap 100. In some implementations, the leading edge 108 may be the preferred end to pass, which may tend to reduce the chance of tearing or binding of the buckle portion 110 of the wrap 100.

In some implementations, once the wrap 100 surrounds the anatomical body 116, the wrap 100 may be positioned such that an edge of the wing 106 is resting at an end 117 of the anatomical body 116. The leading edge 108 may then be inserted through the slit 104 and cinched taut. For example, the wrap 100 may be cinched such that relatively little or no gap remains between the wrap 100 and anatomical body 116, as illustrated in FIG. 1B and FIG. 1C. Further, the leading edge 108 can be seen overlapping and lying flat against the belt portion 105. Additionally, the buckle portion 110 may also overlap and lie flat against the belt portion 105, but in the opposite direction of the leading edge 108. In this arrangement, the amniotic wrap 100 may be secured in such a way that reduces the likelihood of the wrap 100 unraveling or migrating. Also, given the variability of anatomy, any excess length of the wrap 100 may then be trimmed from the first end 101 to a preferred length.

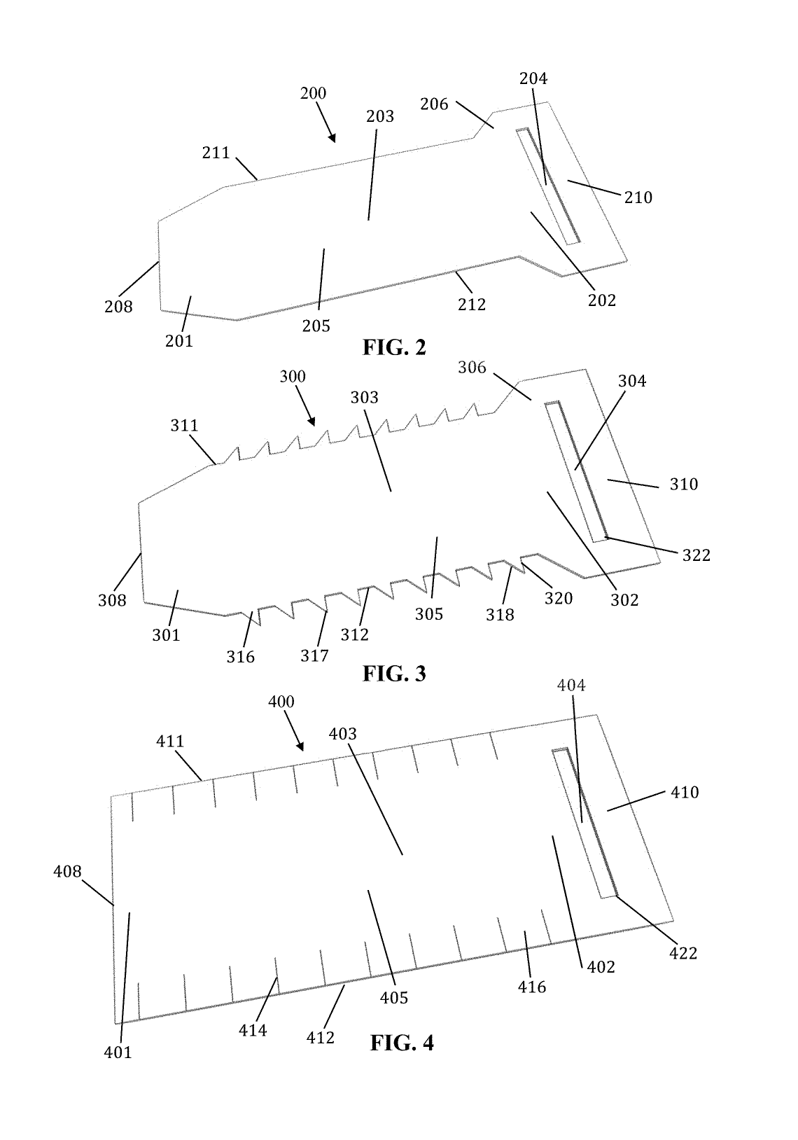

Additional embodiments are shown in FIGS. 2-4, each including variations to the leading edge, the belt portion, the side edges, and the buckle portion, among other things. These figures represent only some of the variations and different arrangements that are contemplated herein. Further, the features and variations discussed herein and shown in the associated figures may be arranged in any combination, according to the particular need.

For instance, FIG. 2 shows an example of an amniotic tissue wrap 200 similar to the wrap 100 shown in FIGS. 1A-1C. The amniotic tissue wrap 200 includes similar features to those discussed above, such as a body 203, first end 201, second end 202, aperture 204, belt portion 205, wings 206, leading edge 208, buckle portion 210, and first and second side edges 211 and 212.

In some embodiments as otherwise discussed herein, and as shown in FIG. 2, the leading edge 208 may include a first width that is narrower than the width of the belt portion 205. Accordingly, the first side edge 211 and second side edge 212 are tapered between the leading edge 208 and the belt portion 205 of the body 203. In some implementations, this may ease the insertion of the leading edge 208 through the aperture 204. Similarly, the width of the body 203 may be tapered between the belt portion 205 and the wings 206 of the buckle portion 210. The amniotic tissue wrap 200 of FIG. 2 may be implanted in substantially the same manner as the tissue wrap 100 discussed above.

FIG. 3 illustrates another embodiment of an amniotic tissue wrap 300, according to another example implementation. Again, the amniotic tissue wrap 300 includes similar features to those discussed above, such as a body 303, first end 301, second end 302, aperture 304, belt portion 305, wings 306, leading edge 308, buckle portion 310, and first and second side edges 311 and 312.

In some embodiments as otherwise discussed herein, and as shown in FIG. 2, the first side edge 311 and the second side edge 312 may each include a plurality of teeth 316 extending laterally from the belt portion 305. Each tooth 316 may include a respective tip 317 that extends laterally wider than the opening width of the aperture 304, which is shown again in FIG. 3 as a slit 304.

Further, each tooth 316 may include a first face 318 that is angled away from the leading edge 308. When the leading edge 308 is passed through the slit 304 while being positioned around the anatomical body 116, as discussed in the examples above, the angled face 318 on the teeth 316 may bend or flex, which may ease the passing of the belt portion 305 through the slit 304, and may reduce the likelihood of one of the teeth 316, the belt portion 305, or the buckle portion 310 tearing.

In some implementation, each tooth 316 may additionally include a second face 320 that is substantially perpendicular to the first side edge 311 and the second side edge 312 of the body 303. After the tissue wrap 300 is cinched taut, as discussed above, the second face 320 of one or more of the teeth 316 may engage with an edge 322 of the aperture 304. In this way, the belt portion 305 and the first end 302 may be discouraged from moving back through the aperture 304, which may further decrease the likelihood of the wrap 300 unraveling.

FIG. 4 illustrates another embodiment of an amniotic tissue wrap 400, according to another example implementation. Again, the amniotic tissue wrap 400 includes similar features to those discussed above, such as a body 403, first end 401, second end 402, aperture 404, belt portion 405, leading edge 408, buckle portion 410, and first and second side edges 411 and 412. In FIG. 4, however, unlike the examples above, the first width of the leading edge 408, the width of the belt portion 405, and the width of the buckle portion 410 are all substantially the same.

In some embodiments, the first side edge 411 and the second side edge 412 of the amniotic tissue wrap 400 may include a plurality of teeth 412 that extend laterally from the belt portion 405. Each tooth 412 may be separated from adjacent teeth by a respective tooth slit 414 formed in the body 403. For example, the tooth slits 414 may be substantially perpendicular to the first side edge 411 and the second side edge 412 of the body 403. Further, the tooth slits 414 may extend from the first side edge 411 and the second side edge 412 inward to the belt portion 405, which may have a resulting belt width that is narrower than the opening width of the aperture 404.

Similar to the example tissue wrap 300 discussed above, when the leading edge 408 is inserted through the aperture 404, the teeth 412 may bend of flex while passing through the aperture 404. After the tissue wrap 400 is cinched taut in a desired position, for instance, in a position surrounding an anatomical body such as the anatomical body 116, one or more of the tooth slits 414 between adjacent teeth 412 may engage an edge 422 of the aperture 404. In this way, similar to the example discussed above, the belt portion 405 and the first end 402 may be discouraged from moving back through the aperture 404, which may further decrease the likelihood of the wrap 400 unraveling.

Although the embodiments and examples discussed herein have generally been presented as an alternative to some traditional methods for securing a tissue graft, such as suturing or applying adhesive, the self-securing biological tissue grafts discussed herein might also be used to improve such traditional methods.

For instance, some of the drawbacks noted above may arise due to the difficulty of maintaining a tissue graft in a desired position while sutures are added or an adhesive is applied. However, the examples tissue grafts discussed herein, and variations thereof, may naturally hold the tissue graft in a desired position, as discussed above. This may facilitate either suturing the tissue graft, or applying an adhesive to it, or both.

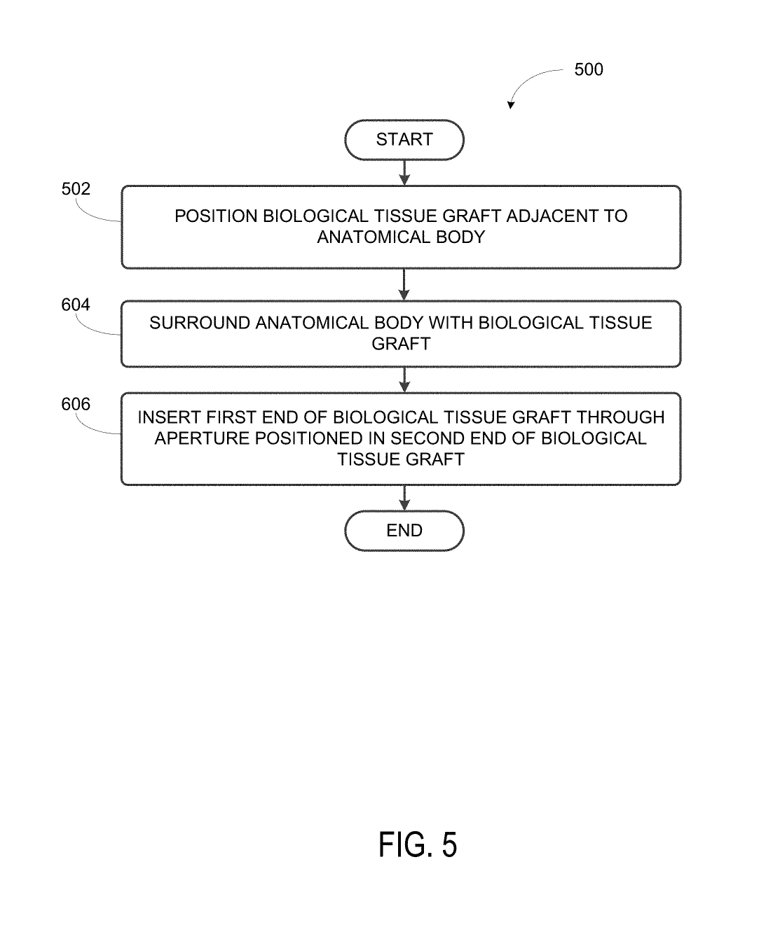

FIG. 5 shows a flowchart of a method 500 for implanting a biological tissue graft, according to an example implementation. Method 500 shown in FIG. 5 presents an embodiment of a method that, for example, could be used with the biological tissue grafts shown in FIGS. 1-4 and discussed above. It should be understood that, for this and other processes and methods disclosed herein, flowcharts show functionality and operation of one possible implementation of present embodiments. In this regard, each block in the flowchart might represent an action taken by a surgeon, for instance. Additionally or alternatively, each block in the flowchart might represent a module, a segment, or a portion of program code, which includes one or more instructions executable by a processor for implementing or causing specific logical functions or steps in the process. For example, the method 500 may be implemented by one or more computing devices of a fully or partially robotic surgical system. Alternative implementations are included within the scope of the example embodiments of the present disclosure, in which functions may be executed out of order from that shown or discussed, including substantially concurrently, depending on the functionality involved, as would be understood by those reasonably skilled in the art.

At block 502, the method 500 includes positioning a biological tissue graft, such as the amniotic tissue wrap 100 as shown in the Figures and discussed above, adjacent to an anatomical body. The method 500 might also be implemented using one of amniotic tissue wraps 200, 300, or 400 as discussed above, or any other biological tissue graft consistent with the embodiments discussed herein. Further, the anatomical body may be similar to the anatomical body 116 shown in FIGS. 1B-1C and discussed above, although the anatomical body may take other shapes or forms as well.

At block 504, the method 500 includes surrounding the anatomical body 116 with the biological tissue graft, as shown by way of example in FIGS. 1B-1C. For instance, surrounding the anatomical body 116 may involve circumferentially wrapping the amniotic tissue wrap 100 around the anatomical body 116 such that the amniotic tissue wrap 100 is flexibly rolled into an approximately cylindrical shape. Other possibilities also exist.

At block 506, the method 500 includes inserting a first end of the biological tissue graft through an aperture positioned in the second end of the biological tissue graft. For example, as shown in FIGS. 1B-1C, the first end 108 of the body 103 of amniotic tissue wrap 100 may be inserted through the aperture 104, which may take the form of a slit. Further, inserting the first end 108 of the amniotic tissue wrap 100 through the aperture 104 may also include cinching the amniotic tissue wrap 100 about the anatomical body 116 so as to secure the amniotic tissue wrap to the anatomical body 116.

In some implementations, where the biological tissue graft includes a plurality of teeth extending laterally from the body, as shown in some of the examples above, inserting the first end of the biological tissue graft through the aperture may include flexibly bending one or more of the plurality of teeth such that the one or more of the plurality of teeth pass through the aperture. For example, as shown in FIG. 3, each of the plurality of teeth 316 may include a leading edge 318 that is angled to facilitate the passing of the teeth 316, and the body 303, of the amniotic tissue wrap 300 through the aperture 304. Further, after inserting the first end 308 through the aperture 304, the method 500 may include engaging at least one of the plurality of teeth 316 with an edge 322 of the aperture 304 such that the first end 308 of the body 303 is discouraged from moving back through the aperture 304.

Some implementations of the method 500 may also include trimming an excess portion of the body from the first end of the body, once the biological tissue graft is implanted at its desired position.

As noted above, the self-securing biological tissue graft may not be implemented as a complete replacement for some of the traditional methods of securing a tissue graft in place during a surgical procedure. Rather, the biological tissue draft herein may be used in conjunction with some traditional practices in order to facilitate implantation, improve the effectiveness of the tissue graft, or a combination of both.

For example, some implementations of the method 500 may include, after inserting the first end of the biological tissue graft through the aperture, securing a portion of the body that has passed through the aperture to an adjacent portion of the body. Securing the body may include one or more of inserting a suture through the body or applying an adhesive to the body, among other possibilities. For instance, referring to FIGS. 1B-1C, once the first end 108 of the amniotic tissue wrap 100 s inserted through the aperture 104, both the first end 108 and the buckle portion 110 of the amniotic tissue wrap 100 lie atop an adjacent portion of the body 103. In one or both of these locations, a suture may be inserted through the adjacent portions of the wrapped body 103.

Advantageously, the belt and buckle engagement of the amniotic tissue wrap 100 may help to hold the amniotic tissue wrap 100 in place while the suture is inserted. In a similar way, an adhesive may be applied between the adjacent portions of the wrapped body 103, at either of both of the locations noted above, while the belt and buckle engagement of the amniotic tissue wrap 100 discourages unravelling. The adhesive may be applied in addition to, or as an alternative to, one or more sutures. Other implementations for securing the biological tissue graft in place are also possible, and may be used in conjunction with the examples discussed herein.

### III. Conclusion

The above discussions relate to a self-securing biological tissue graft and provide only some examples of embodiments and configurations in which the apparatuses and methods described below may be implemented. Other implementations and configurations of self-securing biological tissue grafts not explicitly described herein may also be applicable and suitable for implementation of the apparatuses and methods.

Additionally, references herein to “embodiment” means that a particular feature, structure, or characteristic described in connection with the embodiment can be included in at least one example embodiment of an invention. The appearances of this phrase in various places in the specification are not necessarily all referring to the same embodiment, nor are separate or alternative embodiments mutually exclusive of other embodiments. As such, the embodiments described herein, explicitly and implicitly understood by one skilled in the art, can be combined with other embodiments.

Numerous specific details are set forth to provide a thorough understanding of the present disclosure. However, it is understood to those skilled in the art that certain embodiments of the present disclosure can be practiced without certain, specific details. In other instances, well known methods, procedures, and components have not been described in detail to avoid unnecessarily obscuring aspects of the embodiments. Accordingly, the scope of the present disclosure is defined by the appended claims rather than the foregoing description of embodiments.

We claim:

1. A biological tissue graft comprising:
a body formed from a flexible biological tissue, the body having a first end and a second end opposite the first end; and
an aperture positioned in the second end of the body and sized to receive the first end of the body therethrough.

2. The biological tissue graft of claim 1, wherein the first end of the body comprises a leading edge having a first width, and wherein the aperture is formed as a slit having an opening width that is greater than the first width.

3. The biological tissue graft of claim 2, wherein the body comprises a belt portion positioned between the first end and the second end, wherein the belt portion comprises a belt width.

4. The biological tissue graft of claim 3, wherein the first width of the leading edge is less than the belt width, and wherein the body of the biological tissue graft comprises a first side edge and a second side edge that are tapered between the leading edge and the belt portion of the body.

5. The biological tissue graft of claim 3, wherein the first width of the leading edge and the belt width are substantially the same.

6. The biological tissue graft of claim 3, wherein the second end comprises a buckle portion, the buckle portion including the aperture, and wherein the buckle portion comprises a buckle width that is greater than the belt width.

7. The biological tissue graft of claim 6, wherein the first side edge and the second side edge of the body are tapered between the belt portion of the body and the buckle portion.

8. The biological tissue graft of claim 4, wherein the first side edge and the second side edge of the body each comprise a respective plurality of teeth extending laterally from the belt portion.

9. The biological tissue graft of claim 8, wherein each tooth in the plurality of teeth includes a respective tip, and wherein the respective tip of each tooth in the plurality of teeth extends laterally wider than the opening width of the aperture.

10. The biological tissue graft of claim 8, wherein each tooth in the plurality of teeth comprises a first face angled away from the leading edge of the body and a second face substantially perpendicular to the first side edge and the second side edge of the body.

11. The biological tissue graft of claim 8, wherein each tooth in the plurality of teeth is separated from an adjacent tooth by a respective tooth slit formed in the body, wherein each tooth slit is substantially perpendicular to the first side edge and the second side edge of the body.

12. The biological tissue graft of claim 1, wherein the body is flexibly rollable into an approximately cylindrical shape such that the first end of the body may pass through the aperture.

13. The biological tissue graft of claim 1, wherein the biological tissue graft is formed from amniotic tissue.

14. A method for implanting a biological tissue graft during a surgical procedure, wherein the biological tissue graft comprises a body formed from a flexible biological tissue, the body having a first end, a second end opposite the first end, and an aperture positioned in the second end of the body and sized to receive the first end of the body therethrough, the method comprising:
positioning the biological tissue graft adjacent to an anatomical body;
surrounding the anatomical body with the biological tissue graft; and
inserting the first end of the biological tissue graft through the aperture.

15. The method of claim 14, wherein surrounding the anatomical body with the biological tissue graft comprises circumferentially wrapping the biological tissue graft around the anatomical body such that the biological tissue graft is flexibly rolled into an approximately cylindrical shape.

16. The method of claim 14, wherein inserting the first end of the biological tissue graft through the aperture comprises cinching the biological tissue graft about the anatomical body so as to secure the biological tissue graft to the anatomical body.

17. The method of claim 14, wherein the body of the biological tissue graft comprises a first side edge and a second side edge, wherein the first side edge and the second side edge each comprise a respective plurality of teeth extending laterally from the body, and wherein inserting the first end of the biological tissue graft through the aperture comprises flexibly bending one or more of the plurality of teeth such that the one or more of the plurality of teeth pass through the aperture.

18. The method of claim 17, further comprising:
after inserting the first end of the biological tissue graft through the aperture, engaging at least one of the plurality of teeth with an edge of the aperture such that the first end of the body is discouraged from moving back through the aperture.

19. The method of claim 14, further comprising:
trimming an excess portion of the body from the first end of the body.

20. The method of claim 14, further comprising:
after inserting the first end of the biological tissue graft through the aperture, securing a portion of the body that has passed through the aperture to an adjacent portion of the body.
